(19) United States
(12) Patent Application Publication
(10) Pub. No.: US 2024/0358375 A1
(43) Pub. Date: Oct. 31, 2024

(54) DEVICES AND METHODS FOR LEFT ATRIAL APPENDAGE OCCLUSION

(71) Applicant: Ohio State Innovation Foundation, Columbus, OH (US)

(72) Inventor: Nahush MOKADAM, Upper Arlington, OH (US)

(21) Appl. No.: 18/688,553

(22) PCT Filed: Sep. 2, 2022
(86) PCT No.: PCT/US2022/042503

§ 371 Date: Mar. 1, 2024

Related U.S. Application Data
(60) Provisional application No. 63/240,253, filed on Sep. 2, 2021.

(51) Int. Cl. A61B 17/12; A61B 17/00; A61B 17/122

(52) U.S. Cl. CPC A61B 17/12122; A61B 17/12031; A61B 17/12131; A61B 17/1227; A61B 2017/00367; A61B 2017/00477

(57) ABSTRACT

An occlusion device for occluding a left atrial appendage extending from a lateral wall of a left atrium of a heart may include a first clamping portion and a second clamping portion movably connected to one another and configured for positioning along opposite sides of the left atrial appendage. The first clamping portion and the second clamping portion each may include an inner side configured to face toward the left atrial appendage when the device is in a deployed configuration, an outer side disposed opposite the inner side and configured to face away from the left atrial appendage, a concave side configured to face away from the lateral wall when the device is in the deployed configuration, and a convex side disposed opposite the concave side and configured to face toward the lateral wall.

### CROSS-REFERENCE TO RELATED APPLICATIONS

This application claims the benefit of, and priority to, U.S. Provisional Patent Application No. 63,240,253, filed on Sep. 2, 2021, and titled “Devices and Methods for Left Atrial Appendage Occlusion,” the disclosure of which is expressly incorporated herein by reference in its entirety.

### FIELD OF THE DISCLOSURE

The present disclosure relates generally to devices and methods for occluding a hollow tissue structure of a living subject and more particularly to devices and methods for occluding a left atrial appendage of a subject's heart.

### BACKGROUND OF THE DISCLOSURE

In various instances, a hollow tissue structure of a living subject (i.e., a human or an animal) may be occluded for different medical purposes. For example, a left atrial appendage of a subject's heart often may be occluded, either surgically or percutaneously, for subjects with atrial fibrillation. The left atrial appendage is a hollow tissue structure extending from the lateral wall of the left atrium of the heart. During normal heart function, the left atrial appendage typically contracts along with the remainder of the left atrium, thereby moving blood throughout the hollow space of the left atrial appendage. However, during atrial fibrillation, the left atrial appendage may fail to contract, such that blood may be allowed to pool within the appendage and become stagnated, which may lead to thrombus or clot formation. Such clots may be released from the left atrial appendage into the left atrium and then into the bloodstream, potentially resulting in an obstruction in the subject's brain or other vascular structures and thus increasing risk of stroke. Various techniques may be used to prevent clots from forming within the left atrial appendage and then being released into the patient's bloodstream.

Occlusion of the left atrial appendage is one common approach for decreasing risk of stroke caused by clots formed in the appendage. Left atrial appendage occlusion typically may be performed to prevent blood from entering the appendage and forming clots therein, while also preventing any clots already formed within the appendage from entering the bloodstream. Various types of techniques may be performed, either surgically or percutaneously, to occlude the left atrial appendage of a subject, such as those with atrial fibrillation. Certain techniques use an occlusion device that is formed as a clip, a clamp, or similar structure, which devices are intended to be implanted near the base of the left atrial appendage to isolate the appendage from the subject's blood circulatory system. Examples of this type of occlusion device include the AtriClip device and the AtriClip PRO-V device, both manufactured by AtriCure, Inc. Although such occlusion devices may be suitable in some instances, they may present limitations in certain instances. For example, the use of such occlusion devices may result in incomplete occlusion of the left atrial appendage in certain subjects, which may be attributed to the shape of portions of the occlusion device that engage the left atrial appendage near the base thereof. As discussed further below, the base of the left atrial appendage may have a linear, or substantially linear, shape when the heart is elevated out the subject's chest for implantation of the occlusion device, but the base may assume a non-linear shape when the heart is in situ. The relevant portions of the occlusion device may not be adapted to accommodate this non-linear shape of the base, and thus implantation of the occlusion device may result in incomplete occlusion of the left atrial appendage, leaving a residual stump of the appendage which may lead to thrombus formation and thus subsequent risk of stroke.

A need therefore exists for improved devices and methods for occluding a left atrial appendage of a subject's heart, which may overcome one or more of the above-mentioned limitations associated with existing techniques for left atrial appendage occlusion.

### SUMMARY OF THE DISCLOSURE

The present disclosure provides devices and methods for left atrial appendage occlusion. In one aspect, an occlusion device for occluding a left atrial appendage extending from a lateral wall of a left atrium of a heart is provided. In one embodiment, the occlusion device may include a first clamping portion configured for positioning along a first side of the left atrial appendage, and a second clamping portion movably connected to the first clamping portion and configured for positioning along an opposite second side of the left atrial appendage while the first clamping portion positioned along the first side. In some embodiments, the first clamping portion and the second clamping portion each may include an inner side configured to face toward the left atrial appendage when the occlusion device is in a deployed configuration, an outer side disposed opposite the inner side and configured to face away from the left atrial appendage when the occlusion device is in the deployed configuration, a concave side configured to face away from the lateral wall when the occlusion device is in the deployed configuration, and a convex side disposed opposite the concave side and configured to face toward the lateral wall when the occlusion device is in the deployed configuration.

In some embodiments, the occlusion device also may include a connecting portion connected to the first clamping portion and the second clamping portion and positioned at a first end of the occlusion device, with the second clamping portion being movably connected to the first clamping portion via the connecting portion. In some embodiments, the second clamping portion and the first clamping portion may not be connected to one another at an opposite second end of the occlusion device. In some embodiments, the occlusion device may be configured to transition between an open configuration and a closed configuration. In some embodiments, the connecting portion may be configured to bias the occlusion device toward the closed configuration. In some embodiments, the connecting portion may include a hinge. In some embodiments, the second clamping portion and the first clamping portion may be configured to pivot relative to one another as the occlusion device is transitioned between the open configuration and the closed configuration.

In some embodiments, the occlusion device also may include a first connecting portion connected to the first clamping portion and the second clamping portion and positioned at a first end of the occlusion device, and a second connecting portion connected to the first clamping portion and the second clamping portion and positioned at an opposite second end of the occlusion device, with the second clamping portion being movably connected to the first clamping portion via the first connecting portion and the second connecting portion. In some embodiments, the first clamping portion, the second clamping portion, the first connecting portion, and the second connecting portion may form an enclosed loop defining an opening configured to receive the left atrial appendage therethrough. In some embodiments, the occlusion device may be configured to transition between an open configuration and a closed configuration. In some embodiments, the first connecting portion and the second connecting portion may be configured to bias the occlusion device toward the closed configuration. In some embodiments, the first connecting portion and the second connecting portion each may have a curved shape. In some embodiments, the second clamping portion and the first clamping portion may be configured to translate relative to one another as the occlusion device is transitioned between the open configuration and the closed configuration.

In some embodiments, the inner side may include a planar surface having a linear profile viewed from a top side or a bottom side of the occlusion device. In some embodiments, the inner side may include a contoured surface having a contoured profile viewed from a top side or a bottom side of the occlusion device. In some embodiments, the outer side may include a planar surface having a linear profile viewed from a top side or a bottom side of the occlusion device. In some embodiments, the outer side may include a contoured surface having a contoured profile viewed from a top side or a bottom side of the occlusion device. In some embodiments, the concave side may include a concave surface having a concave profile viewed from a lateral side of the occlusion device. In some embodiments, the concave surface may extend along at least a majority of a length of the clamping portion. In some embodiments, the concave surface may extend along an entirety of the length of the clamping portion. In some embodiments, the convex side may include a convex surface having a convex profile viewed from a lateral side of the occlusion device. In some embodiments, the convex surface may extend along at least a majority of a length of the clamping portion. In some embodiments, the convex surface may extend along an entirety of the length of the clamping portion. In some embodiments, the occlusion device also may include a fabric covering extending over at least a portion of each of the first clamping portion and the second clamping portion. In some embodiments, the fabric covering may extend over at least a portion of each of the inner side, the outer side, the concave side, and the convex side.

In another aspect, a method for occluding a left atrial appendage extending from a lateral wall of a left atrium of a heart is provided. In one embodiment, the method may include causing an occlusion device to transition from a closed configuration to an open configuration. In some embodiments, the occlusion device may include a first clamping portion and a second clamping portion movably connected to the first clamping portion. In some embodiments, the first clamping portion and the second clamping portion each may include an inner side, an outer side disposed opposite the inner side, a concave side, and a convex side disposed opposite the concave side. In some embodiments, the method also may include positioning the occlusion device relative to the left atrial appendage such that the first clamping portion is positioned along a first side of the left atrial appendage and the second clamping portion is positioned along an opposite second side of the left atrial appendage, and causing the occlusion device to transition from the open configuration to a deployed configuration in which the occlusion device occludes the left atrial appendage. In some embodiments, when the occlusion device is in the deployed configuration, the inner sides of the first clamping portion and the second clamping portion each may face toward the left atrial appendage, the outer sides of the first clamping portion and the second clamping portion each may face away from the left atrial appendage, the concave sides of the first clamping portion and the second clamping portion each may face away from the lateral wall, and the convex sides of the first clamping portion and the second clamping portion each may face toward the lateral wall.

In some embodiments, the occlusion device also may include a connecting portion connected to the first clamping portion and the second clamping portion and positioned at a first end of the occlusion device, with the second clamping portion being movably connected to the first clamping portion via the connecting portion. In some embodiments, the second clamping portion and the first clamping portion may not be connected to one another at an opposite second end of the occlusion device. In some embodiments, causing the occlusion device to transition from the closed configuration to the open configuration may include causing the first clamping portion and the second clamping portion to pivot away from one another. In some embodiments, the connecting portion may be configured to bias the occlusion device toward the closed configuration. In some embodiments, causing the occlusion device to transition from the closed configuration to the open configuration may include applying an external force to the occlusion device to overcome a biasing force provided by the connecting portion. In some embodiments, causing the occlusion device to transition from the open configuration to the deployed configuration may include removing the external force. In some embodiments, causing the occlusion device to transition from the open configuration to the deployed configuration may include allowing the connecting portion to bias the occlusion device to the deployed configuration. In some embodiments, the connecting portion may include a hinge.

In some embodiments, the occlusion device also may include a first connecting portion connected to the first clamping portion and the second clamping portion and positioned at a first end of the occlusion device, and a second connecting portion connected to the first clamping portion and the second clamping portion and positioned at an opposite second end of the occlusion device, with the second clamping portion being movably connected to the first clamping portion via the first connecting portion and the second connecting portion. In some embodiments, the first clamping portion, the second clamping portion, the first connecting portion, and the second connecting portion may form an enclosed loop defining an opening, and positioning the occlusion device relative to the left atrial appendage may include positioning the occlusion device such that the left atrial appendage is received through the opening. In some embodiments, causing the occlusion device to transition from the closed configuration to the open configuration may include causing the first clamping portion and the second clamping portion to translate away from one another. In some embodiments, the first connecting portion and the second connecting portion may be configured to bias the occlusion device toward the closed configuration. In some embodiments, causing the occlusion device to transition from the closed configuration to the open configuration may include applying an external force to the occlusion device to overcome a biasing force provided by the first connecting portion and the second connecting portion. In some embodiments, causing the occlusion device to transition from the open configuration to the deployed configuration may include removing the external force. In some embodiments, causing the occlusion device to transition from the open configuration to the deployed configuration may include allowing the first connecting portion and the second connecting portion to bias the occlusion device to the deployed configuration. In some embodiments, the first connecting portion and the second connecting portion each may have a curved shape. In some embodiments, causing the occlusion device to transition from the closed configuration to the open configuration may include causing the first clamping portion and the second clamping portion to pivot away from one another.

In some embodiments, the inner side may include a planar surface having a linear profile viewed from a top side or a bottom side of the occlusion device. In some embodiments, the inner side may include a contoured surface having a contoured profile viewed from a top side or a bottom side of the occlusion device. In some embodiments, the outer side may include a planar surface having a linear profile viewed from a top side or a bottom side of the occlusion device. In some embodiments, the outer side may include a contoured surface having a contoured profile viewed from a top side or a bottom side of the occlusion device. In some embodiments, the concave side may include a concave surface having a concave profile viewed from a lateral side of the occlusion device. In some embodiments, the concave surface may extend along at least a majority of a length of the clamping portion. In some embodiments, the concave surface may extend along an entirety of the length of the clamping portion. In some embodiments, the convex side may include a convex surface having a convex profile viewed from a lateral side of the occlusion device. In some embodiments, the convex surface may extend along at least a majority of a length of the clamping portion. In some embodiments, the convex surface may extend along an entirety of the length of the clamping portion. In some embodiments, the occlusion device also may include a fabric covering extending over at least a portion of each of the first clamping portion and the second clamping portion. In some embodiments, the fabric covering may extend over at least a portion of each of the inner side, the outer side, the concave side, and the convex side.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a perspective view of a portion of a heart while the heart is in situ, showing a left atrial appendage extending from a lateral wall of a left atrium of the heart, with a base of the left atrial appendage having a non-linear shape. FIG. 1B is a cross-sectional view of the portion of the heart of FIG. 1A while the heart is in situ, with the cross-section taken along plane 1B-1B of FIG. 1A. FIG. 1C is a perspective view of the portion of the heart of FIG. 1A while the heart is elevated from the chest, showing the base of the left atrial appendage having a linear shape.

FIG. 2A is a top view of an example occlusion device in a closed configuration. FIG. 2B is a side view of the occlusion device of FIG. 2A in the closed configuration. FIG. 2C is a top view of the occlusion device of FIG. 2A in an open configuration. FIG. 2D is a side view of the occlusion device of FIG. 2A in the open configuration. FIG. 2E is a perspective view of the occlusion device of FIG. 2A in a deployed configuration, showing the occlusion device secured to the left atrial appendage of the portion of the heart of FIG. 1A while the heart is elevated from the chest. FIG. 2F is a perspective view of the occlusion device of FIG. 2A in the deployed configuration, showing the occlusion device secured to the left atrial appendage of the portion of the heart of FIG. 1A while the heart is in situ.

FIG. 3A is a top view of an example occlusion device in a closed configuration. FIG. 3B is a side view of the occlusion device of FIG. 3A in the closed configuration. FIG. 3C is an end view of the occlusion device of FIG. 3A in the closed configuration. FIG. 3D is a top view of the occlusion device of FIG. 3A in an open configuration. FIG. 3E is a side view of the occlusion device of FIG. 3A in the open configuration. FIG. 3F is an end view of the occlusion device of FIG. 3A in the open configuration.

FIG. 4A is a top view of an example occlusion device in accordance with one or more embodiments of the disclosure, showing the occlusion device in a closed configuration. FIG. 4B is a side view of the occlusion device of FIG. 4A in the closed configuration. FIG. 4C is a top view of the occlusion device of FIG. 4A in an open configuration. FIG. 4D is a side view of the occlusion device of FIG. 4A in the open configuration. FIG. 4E is a perspective view of the occlusion device of FIG. 4A in a deployed configuration, showing the occlusion device secured to the left atrial appendage of the portion of the heart of FIG. 1A while the heart is elevated from the chest. FIG. 4F is a perspective view of the occlusion device of FIG. 4A in the deployed configuration, showing the occlusion device secured to the left atrial appendage of the portion of the heart of FIG. 1A while the heart is in situ.

FIG. 5A is a top view of an example occlusion device in accordance with one or more embodiments of the disclosure, showing the occlusion device in a closed configuration. FIG. 5B is a side view of the occlusion device of FIG. 5A in the closed configuration. FIG. 5C is an end view of the occlusion device of FIG. 5A in the closed configuration. FIG. 5D is a top view of the occlusion device of FIG. 5A in an open configuration. FIG. 5E is a side view of the occlusion device of FIG. 5A in the open configuration. FIG. 5F is an end view of the occlusion device of FIG. 5A in the open configuration.

FIG. 6A is a top view of an example occlusion device in accordance with one or more embodiments of the disclosure, showing the occlusion device in a closed configuration. FIG. 6B is a side view of the occlusion device of FIG. 6A in the closed configuration. FIG. 6C is an end view of the occlusion device of FIG. 6A in the closed configuration. FIG. 6D is a top view of the occlusion device of FIG. 6A in an open configuration. FIG. 6E is a side view of the occlusion device of FIG. 6A in the open configuration. FIG. 6F is an end view of the occlusion device of FIG. 6A in the open configuration.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and components have not been described in detail so as not to unnecessarily obscure aspects of the embodiments.

### Overview

Embodiments of devices and methods for left atrial appendage occlusion are provided herein. According to disclosed embodiments, an occlusion device generally may include a first clamping portion and a second clamping portion configured for positioning along opposite sides of the left atrial appendage. The second clamping portion may be movably connected to the first clamping portion, and the occlusion device may be configured to transition between an open configuration and a closed configuration. In this manner, the occlusion device may be introduced and positioned with respect to the left atrial appendage while the device is in the open configuration. Upon proper positioning of the occlusion device, the device may be transitioned from the open configuration toward the closed configuration. Specifically, the occlusion device may be transitioned from the open configuration to a deployed configuration in which the device occludes the left atrial appendage.

According to disclosed embodiments, the first clamping portion and the second clamping portion each may include an inner side, an outer side, a concave side, and a convex side. When the occlusion device is in the deployed configuration, the inner side may face toward the left atrial appendage, the outer side may face away from the left atrial appendage, the concave side may face away from the lateral wall of the left atrium from which the left atrial appendage extends, and the convex side may face toward the lateral wall. As described below, the shape of the first clamping portion and the second clamping portion of the occlusion device advantageously may decrease incidence of incomplete occlusion as compared to certain existing occlusion devices. In particular, the shapes of the concave sides and the convex sides of the first clamping portion and the second clamping portion may better accommodate the shape of the base of the left atrial appendage in a manner that avoids a residual stump remaining un-occluded. According to various embodiments, the radius of curvature and the depth of curvature of the concave sides and the convex sides may be varied to better accommodate the anatomy of different subjects.

Still other benefits and advantages of the occlusion devices and methods provided herein over existing technology for left atrial appendage occlusion will be appreciated by those of ordinary skill in the art from the following description and the appended drawings.

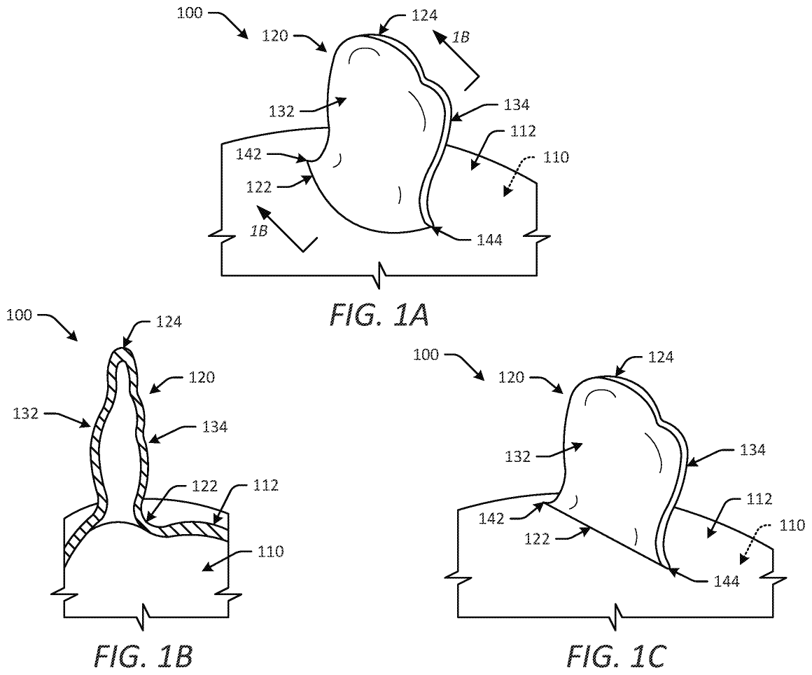

Referring now to FIGS. 1A-1C, a portion of a heart 100 of a living subject (i.e., a human or an animal) is depicted. FIGS. 1A and 1B show the portion of the heart 100 while the heart 100 is in situ. FIG. 1C shows the portion of the heart 100 while the heart 100 is elevated from the chest of the subject, which positioning often may be used when performing a left atrial appendage occlusion procedure. As shown, a left atrial appendage 120 may extend from a lateral wall 112 of a left atrium 110 of the heart 100. It will be appreciated that the shape and size of the illustrated left atrial appendage 120 is merely an example, as the shape and size of the tissue structure may vary from one subject to another. As shown, the left atrial appendage 120 may include a base 122 and a tip 124 disposed opposite one another, with the base 122 connected to the lateral wall 112. The left atrial appendage 120 may have an overall height extending from the base 122 to the tip 124. As shown, the left atrial appendage 120 may include a first side 132 and a second side 134 disposed opposite one another, with the left atrial appendage 120 having an overall width extending from the first side 132 to the second side 134. The left atrial appendage 120 may include a first end 142 and a second end 144 disposed opposite one another, with the left atrial appendage 120 having an overall length extending from the first end 142 to the second end 144.

As shown, the shape of the base 122 of the left atrial appendage 120 may be different when the heart 100 is elevated from the chest as compared to when the heart 100 is in situ. In particular, the present inventor has observed that, while the heart 100 is elevated, the base 122 may have a linear, or substantially linear, shape, as shown in FIG. 1C, and that, while the heart 100 is in situ, the base 122 may have a non-linear shape, as shown in FIG. 1A. The non-linear shape of the base 122 may be generally C-shaped, with a radius of curvature and a depth of curvature of the base 122 varying from one subject to another. The non-linear shape of the base 122 is due to the positioning of the left atrial appendage 120 at the reflection of the left superior pulmonary vein and the body of the left atrium 110, which forms a fold. The present inventor has observed that, when performing an occlusion procedure with certain existing occlusion devices, the non-linear shape of the base 122 may result in incomplete occlusion of the left atrial appendage 120 in a varying number of subjects, depending on the series and the degree to which this is evaluated by imaging. Such incomplete occlusion may leave a residual stump of the left atrial appendage 120, which may be a source of thrombus formation and thus may present a subsequent risk of stroke. It is estimated that procedures using certain existing occlusion devices may result in incomplete occlusion in as many as 25% of cases.

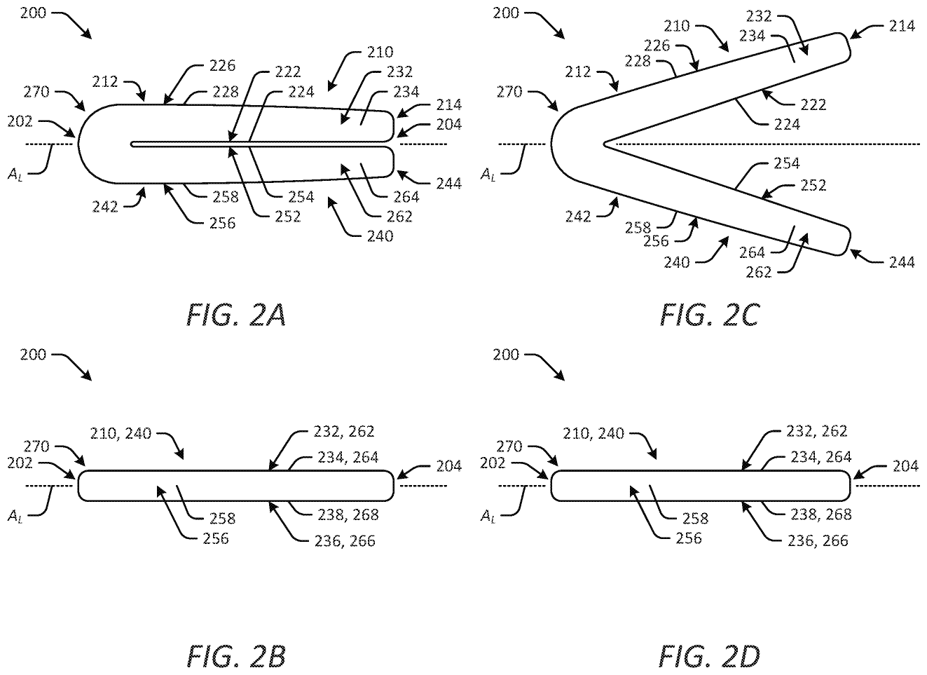

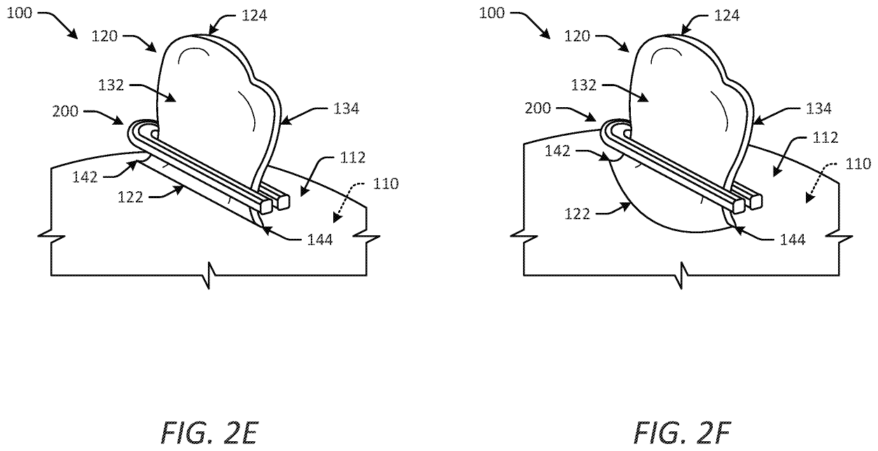

FIGS. 2A-2F illustrate an example occlusion device 200, which generally may be representative of certain existing occlusion devices. The occlusion device 200 may be formed as an elongate structure, with a first end 202 and a second end 204 disposed opposite one another along a longitudinal axis A_{L} of the device 200. As shown, the device 200 may include a first clamping portion 210 and a second clamping portion 240 connected to one another by a connecting portion 270. The connecting portion 270 may be configured to allow the first clamping portion 210 and the second clamping portion 240 to move relative to one another. Specifically, the occlusion device 200 may be configured to transition between a closed configuration, as shown in FIGS. 2A and 2B, and an open configuration, as shown in FIGS. 2C and 2D. The device 200 may be biased from the open configuration toward, or to, the closed configuration. For example, the connecting portion 270 may bias the device 200 from the open configuration toward, or to, the closed configuration. As shown, the connecting portion 270 may be formed as a hinge and configured to allow the first clamping portion 210 and the second clamping portion 240 to pivot relative to one another as the device 200 transitions between the closed configuration and the open configuration.

The first clamping portion 210 may be formed as an elongate structure, with a first end 212 and a second end 214 disposed opposite one another. The first clamping portion 210 may extend from the connecting portion 270 to the second end 204 of the device 200. As shown, the first clamping portion 210 may include an inner side 222 having an inner surface 224, an outer side 226 disposed opposite the inner side 222 and having an outer surface 228, a top side 232 having a top surface 234, and a bottom side 236 disposed opposite the top side 232 and having a bottom surface 238. As shown, the inner surface 224 may be a planar surface having a linear profile when viewed from the top side or the bottom side of the device 200, while the outer surface 228 may be a slightly contoured surface having a non-linear profile when viewed from the top side or the bottom side of the device 200. As shown, the top surface 234 may be a planar surface having a linear profile when viewed from one of the lateral sides of the device 200, and the bottom surface 238 may be a planar surface having a linear profile when viewed from one of the lateral sides of the device 200.

Similarly, the second clamping portion 240 may be formed as an elongate structure, with a first end 242 and a second end 244 disposed opposite one another. The second clamping portion 240 may extend from the connecting portion 270 to the second end 204 of the device 200. As shown, the second clamping portion 240 may include an inner side 252 having an inner surface 254, an outer side 256 disposed opposite the inner side 252 and having an outer surface 258, a top side 262 having a top surface 264, and a bottom side 266 disposed opposite the top side 262 and having a bottom surface 268. As shown, the inner surface 254 may be a planar surface having a linear profile when viewed from the top side or the bottom side of the device 200, while the outer surface 258 may be a slightly contoured surface having a non-linear profile when viewed from the top side or the bottom side of the device 200. As shown, the top surface 264 may be a planar surface having a linear profile when viewed from one of the lateral sides of the device 200, and the bottom surface 268 may be a planar surface having a linear profile when viewed from one of the lateral sides of the device 200. As shown, the second clamping portion 240 may be formed as a mirror image of the first clamping portion 210 across a plane extending through the longitudinal axis A_{L} of the device 200.

During use, the occlusion device 200 may be introduced and positioned relative to a left atrial appendage while the device 200 is in the open configuration. Upon positioning, the device 200 may be transitioned from the open configuration toward the closed configuration. It will be appreciated that, with the left atrial appendage positioned between the first clamping portion 210 and the second clamping portion 240, the appendage may inhibit the device 200 from transitioning all the way to the closed configuration shown in FIGS. 2A and 2B. Rather, the device 200 may transition to a deployed configuration, as shown in FIGS. 2E and 2F. As shown, one of the clamping portions 210, 240 may be positioned along the first side 132 of the left atrial appendage 120, while the other clamping portion 210, 240 may be positioned along the second side 134 of the left atrial appendage 120. When the device 200 is in the deployed configuration, the inner sides 222, 252 of the clamping portions 210, 240 may face toward the left atrial appendage 120, the outer sides 226, 256 of the clamping portions 210, 240 may face away from the left atrial appendage 120, the top sides 232, 262 of the clamping portions 210, 240 may face away from the lateral wall 112 of the left atrium 110, and the bottom sides 236, 266 of the clamping portions 210, 240 may face toward the lateral wall 112 of the left atrium 110.

FIGS. 2E and 2F illustrate the problem of incomplete occlusion of the left atrial appendage 120 due to the non-linear shape of the base 122 when the heart 100 is in situ. FIG. 2E illustrates the portion of the heart 100 while the heart 100 is elevated during implantation of the device 200, showing the base 122 having a linear, or substantially linear, shape. FIG. 2F illustrates the portion of the heart 100 while the heart 100 is in situ, showing the base 122 having a non-linear shape. As shown, the non-linear shape of the base 122 may result in incomplete occlusion of the left atrial appendage 120 when using the occlusion device 200, leaving a residual stump of the left atrial appendage 120.

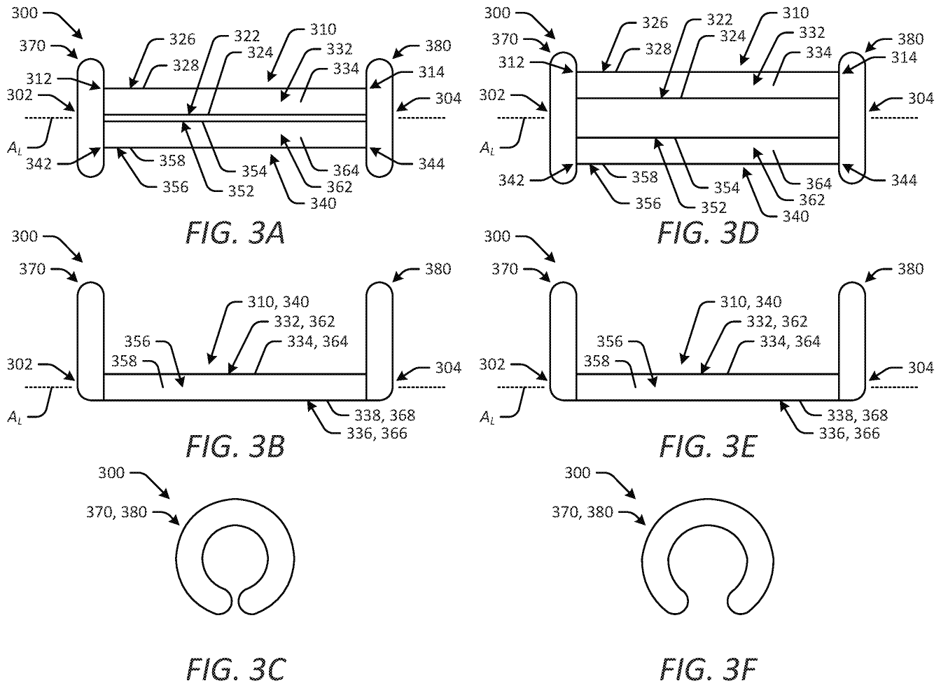

FIGS. 3A-3F illustrate an example occlusion device 300, which generally may be representative of certain existing occlusion devices. The occlusion device 300 may be formed as an elongate structure, with a first end 302 and a second end 304 disposed opposite one another along a longitudinal axis A_{L} of the device 300. As shown, the device 300 may include a first clamping portion 310 and a second clamping portion 340 connected to one another by each of a first connecting portion 370 and a second connecting portion 380. The first connecting portion 370 and the second connecting portion 380 may be configured to allow the first clamping portion 310 and the second clamping portion 340 to move relative to one another. Specifically, the occlusion device 300 may be configured to transition between a closed configuration, as shown in FIGS. 3A-3C, and an open configuration, as shown in FIGS. 3D-3F. The device 300 may be biased from the open configuration toward, or to, the closed configuration. For example, the connecting portions 370, 380 may bias the device 300 from the open configuration toward, or to, the closed configuration. As shown, the connecting portions 370, 380 may be formed as curved members configured to allow the first clamping portion 310 and the second clamping portion 340 to translate relative to one another as the device 300 transitions between the closed configuration and the open configuration.

The first clamping portion 310 may be formed as an elongate structure, with a first end 312 and a second end 314 disposed opposite one another. The first clamping portion 310 may extend from the first connecting portion 370 to the second connecting portion 380. As shown, the first clamping portion 310 may include an inner side 322 having an inner surface 324, an outer side 326 disposed opposite the inner side 322 and having an outer surface 328, a top side 332 having a top surface 334, and a bottom side 336 disposed opposite the top side 332 and having a bottom surface 338. As shown, the inner surface 324 may be a curved surface having a linear profile when viewed from the top side or the bottom side of the device 300, and the outer surface 328 may be a curved surface having a linear profile when viewed from the top side or the bottom side of the device 300. As shown, the top surface 334 may be a curved surface having a linear profile when viewed from one of the lateral sides of the device 300, and the bottom surface 338 may be a curved surface having a linear profile when viewed from one of the lateral sides of the device 300.

Similarly, the second clamping portion 340 may be formed as an elongate structure, with a first end 342 and a second end 344 disposed opposite one another. The second clamping portion 340 may extend from the first connecting portion 370 to the second connecting portion 380. As shown, the second clamping portion 340 may include an inner side 352 having an inner surface 354, an outer side 356 disposed opposite the inner side 352 and having an outer surface 358, a top side 362 having a top surface 364, and a bottom side 366 disposed opposite the top side 362 and having a bottom surface 368. As shown, the inner surface 354 may be a curved surface having a linear profile when viewed from the top side or the bottom side of the device 300, and the outer surface 358 may be a curved having a linear profile when viewed from the top side or the bottom side of the device 300. As shown, the top surface 364 may be a curved surface having a linear profile when viewed from one of the lateral sides of the device 300, and the bottom surface 368 may be a curved surface having a linear profile when viewed from one of the lateral sides of the device 300. As shown, the second clamping portion 340 may be formed as a mirror image of the first clamping portion 310 across a plane extending through the longitudinal axis A_{L} of the device 300.

During use, the occlusion device 300 may be introduced and positioned relative to a left atrial appendage while the device 300 is in the open configuration. Upon positioning, the device 300 may be transitioned from the open configuration toward the closed configuration. It will be appreciated that, with the left atrial appendage positioned between the first clamping portion 310 and the second clamping portion 340, the appendage may inhibit the device 300 from transitioning all the way to the closed configuration shown in FIGS. 3A-3C. Rather, the device 300 may transition to a deployed configuration between the open configuration and the closed configuration. One of the clamping portions 310, 340 may be positioned along the first side 132 of the left atrial appendage 120, while the other clamping portion 310, 340 may be positioned along the second side 134 of the left atrial appendage 120. When the device 300 is in the deployed configuration, the inner sides 322, 352 of the clamping portions 310, 340 may face toward the left atrial appendage 120, the outer sides 326, 356 of the clamping portions 310, 340 may face away from the left atrial appendage 120, the top sides 332, 362 of the clamping portions 310, 340 may face away from the lateral wall 112 of the left atrium 110, and the bottom sides 336, 366 of the clamping portions 310, 340 may face toward the lateral wall 112 of the left atrium 110.

It will be appreciated that use of the occlusion device 300, similar to the occlusion device 200 described above, also may result in the problem of incomplete occlusion of the left atrial appendage 120 due to the non-linear shape of the base 122 when the heart 100 is in situ, leaving a residual stump of the left atrial appendage 120.

### Example Embodiments of Occlusion Devices

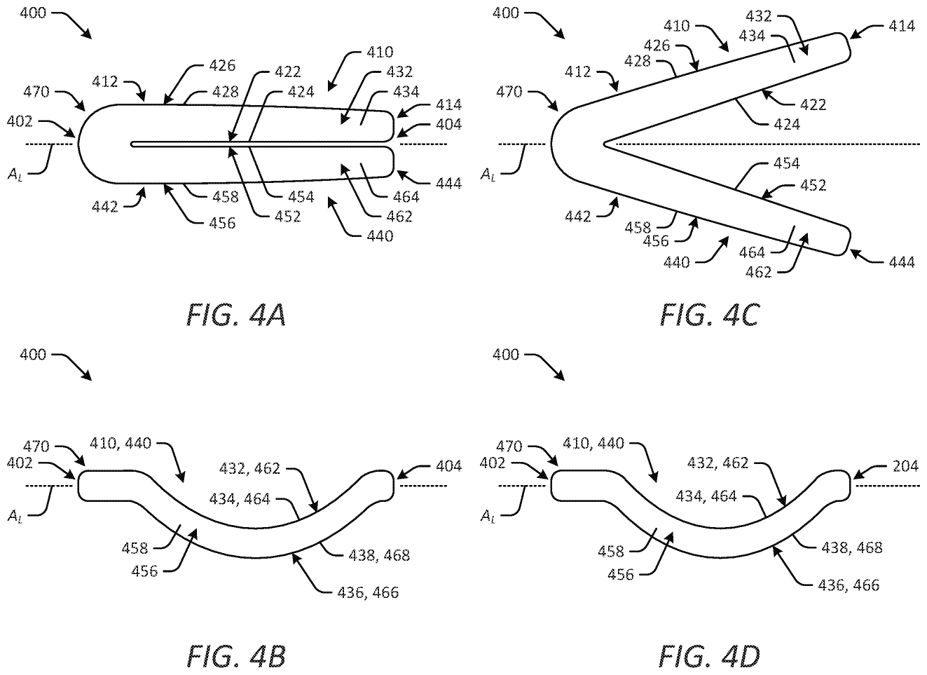

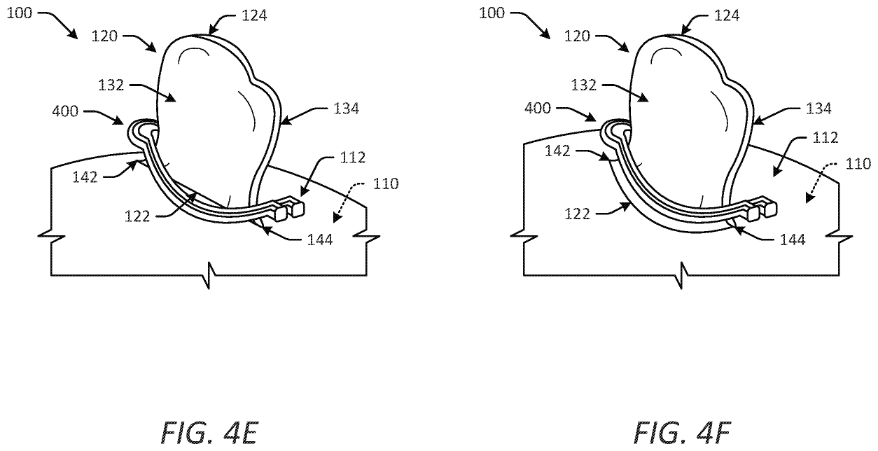

FIGS. 4A-4F illustrate an example occlusion device 400 (which also may be referred to as a “left atrial appendage occlusion device,” a “device,” a “clamp,” a “clip,” or an “implant”) in accordance with embodiments of the disclosure. The occlusion device 400 may be formed as an elongate structure, with a first end 402 and a second end 404 disposed opposite one another along a longitudinal axis A_{L} of the device 400. As shown, the device 400 may include a first clamping portion 410 and a second clamping portion 440 connected to one another by a connecting portion 470. The connecting portion 470 may be configured to allow the first clamping portion 410 and the second clamping portion 440 to move relative to one another. Specifically, the occlusion device 400 may be configured to transition between a closed configuration, as shown in FIGS. 4A and 4B, and an open configuration, as shown in FIGS. 4C and 4D. The device 400 may be biased from the open configuration toward, or to, the closed configuration. For example, the connecting portion 470 may bias the device 400 from the open configuration toward, or to, the closed configuration. As shown, the connecting portion 470 may be formed as a hinge and configured to allow the first clamping portion 410 and the second clamping portion 440 to pivot relative to one another as the device 400 transitions between the closed configuration and the open configuration.

The first clamping portion 410 may be formed as an elongate structure, with a first end 412 and a second end 414 disposed opposite one another. The first clamping portion 410 may extend from the connecting portion 470 to the second end 404 of the device 400. As shown, the first clamping portion 410 may include an inner side 422 having an inner surface 424, an outer side 426 disposed opposite the inner side 422 and having an outer surface 428, a top side 432 (which also may be referred to as a “concave side”) having a top surface 434 (which also may be referred to as a “concave surface”), and a bottom side 436 (which also may be referred to as a “convex side”) disposed opposite the top side 432 and having a bottom surface 438 (which also may be referred to as a “convex side”). As shown, the inner surface 424 may be a planar surface having a linear profile when viewed from the top side or the bottom side of the device 400, while the outer surface 428 may be a slightly contoured surface having a non-linear profile when viewed from the top side or the bottom side of the device 400. As shown, the top surface 434 may be a concave surface having a concave profile when viewed from one of the lateral sides of the device 400, and the bottom surface 438 may be a convex surface having a convex profile when viewed from one of the lateral sides of the device 400. According to various embodiments, the radius of curvature and the depth of curvature of the top surface 434 and the bottom surface 438 may be varied to better accommodate the anatomy of different subjects.

Similarly, the second clamping portion 440 may be formed as an elongate structure, with a first end 442 and a second end 444 disposed opposite one another. The second clamping portion 440 may extend from the connecting portion 470 to the second end 404 of the device 400. As shown, the second clamping portion 440 may include an inner side 452 having an inner surface 454, an outer side 456 disposed opposite the inner side 452 and having an outer surface 458, a top side 462 (which also may be referred to as a “concave side”) having a top surface 464 (which also may be referred to as a “concave surface”), and a bottom side 466 (which also may be referred to as a “convex side”) disposed opposite the top side 462 and having a bottom surface 468 (which also may be referred to as a “convex surface”). As shown, the inner surface 454 may be a planar surface having a linear profile when viewed from the top side or the bottom side of the device 400, while the outer surface 458 may be a slightly contoured surface having a non-linear profile when viewed from the top side or the bottom side of the device 400. As shown, the top surface 464 may be a concave surface having a concave profile when viewed from one of the lateral sides of the device 400, and the bottom surface 468 may be a convex surface having a convex profile when viewed from one of the lateral sides of the device 400. According to various embodiments, the radius of curvature and the depth of curvature of the top surface 464 and the bottom surface 468 may be varied to better accommodate the anatomy of different subjects. As shown, the second clamping portion 440 may be formed as a mirror image of the first clamping portion 410 across a plane extending through the longitudinal axis A_{L} of the device 400.

During use, the occlusion device 400 may be introduced and positioned relative to a left atrial appendage while the device 400 is in the open configuration. Upon positioning, the device 400 may be transitioned from the open configuration toward the closed configuration. It will be appreciated that, with the left atrial appendage positioned between the first clamping portion 410 and the second clamping portion 440, the appendage may inhibit the device 400 from transitioning all the way to the closed configuration shown in FIGS. 4A and 4B. Rather, the device 400 may transition to a deployed configuration, as shown in FIGS. 4E and 4F. As shown, one of the clamping portions 410, 440 may be positioned along the first side 132 of the left atrial appendage 120, while the other clamping portion 410, 440 may be positioned along the second side 134 of the left atrial appendage 120. When the device 400 is in the deployed configuration, the inner sides 422, 452 of the clamping portions 410, 440 may face toward the left atrial appendage 120, the outer sides 426, 456 of the clamping portions 410, 440 may face away from the left atrial appendage 120, the top sides 432, 462 of the clamping portions 410, 440 may face away from the lateral wall 112 of the left atrium 110, and the bottom sides 436, 466 of the clamping portions 410, 440 may face toward the lateral wall 112 of the left atrium 110.

FIGS. 4E and 4F illustrate how the shapes of the clamping portions 410, 440 of the occlusion device 400 may alleviate the problem of incomplete occlusion of the left atrial appendage 120 due to the non-linear shape of the base 122 when the heart 100 is in situ. FIG. 4E illustrates the portion of the heart 100 while the heart 100 is elevated during implantation of the device 400, showing the base 122 having a linear, or substantially linear, shape. FIG. 4F illustrates the portion of the heart 100 while the heart 100 is in situ, showing the base 122 having a non-linear shape. As compared to the clamping portions 210, 240 of the device 200 described above, the shapes of the clamping portions 410, 440 of the device 400 may better accommodate the non-linear shape of the base 122, reducing incidence of incomplete occlusion of the left atrial appendage 120 when using the occlusion device 400.

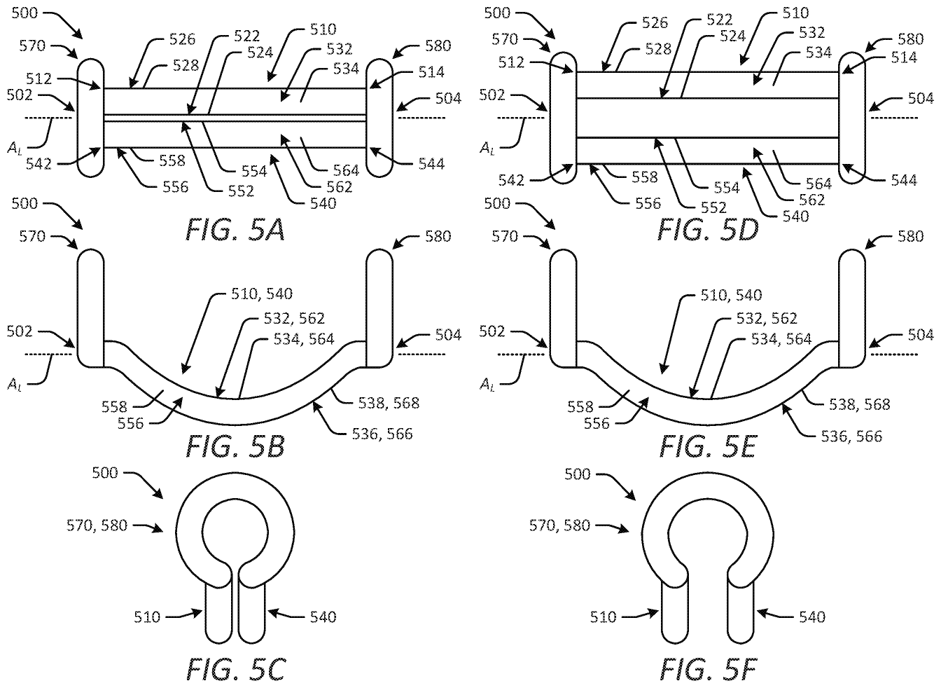

FIGS. 5A-5F illustrate another example occlusion device 500 (which also may be referred to as a “left atrial appendage occlusion device,” a “device,” a “clamp,” a “clip,” or an “implant”) in accordance with embodiments of the disclosure. Certain similarities and differences between the occlusion device 500 and the occlusion device 400 described above will be appreciated from the following description and the respective drawings. The occlusion device 500 may be formed as an elongate structure, with a first end 502 and a second end 504 disposed opposite one another along a longitudinal axis A_{L} of the device 500. As shown, the device 500 may include a first clamping portion 510 and a second clamping portion 540 connected to one another by each of a first connecting portion 570 and a second connecting portion 580. The first connecting portion 570 and the second connecting portion 580 may be configured to allow the first clamping portion 510 and the second clamping portion 540 to move relative to one another. Specifically, the occlusion device 500 may be configured to transition between a closed configuration, as shown in FIGS. 5A-5C, and an open configuration, as shown in FIGS. 5D-5F. The device 500 may be biased from the open configuration toward, or to, the closed configuration. For example, the connecting portions 570, 580 may bias the device 500 from the open configuration toward, or to, the closed configuration. As shown, the connecting portions 570, 580 may be formed as curved members configured to allow the first clamping portion 510 and the second clamping portion 540 to translate relative to one another as the device 500 transitions between the closed configuration and the open configuration.

The first clamping portion 510 may be formed as an elongate structure, with a first end 512 and a second end 514 disposed opposite one another. The first clamping portion 510 may extend from the first connecting portion 570 to the second connecting portion 580. As shown, the first clamping portion 510 may include an inner side 522 having an inner surface 524, an outer side 526 disposed opposite the inner side 522 and having an outer surface 528, a top side 532 (which also may be referred to as a “concave side”) having a top surface 534 (which also may be referred to as a “concave surface”), and a bottom side 536 (which also may be referred to as a “convex side”) disposed opposite the top side 532 and having a bottom surface 538 (which also may be referred to as a “convex surface”). As shown, the inner surface 524 may be a curved surface having a linear profile when viewed from the top side or the bottom side of the device 500, and the outer surface 528 may be a curved surface having a linear profile when viewed from the top side or the bottom side of the device 500. As shown, the top surface 534 may be a curved surface having a concave profile when viewed from one of the lateral sides of the device 500, and the bottom surface 538 may be a curved surface having a convex profile when viewed from one of the lateral sides of the device 500. According to various embodiments, the radius of curvature and the depth of curvature of the top surface 534 and the bottom surface 538 may be varied to better accommodate the anatomy of different subjects.

Similarly, the second clamping portion 540 may be formed as an elongate structure, with a first end 542 and a second end 544 disposed opposite one another. The second clamping portion 540 may extend from the first connecting portion 570 to the second connecting portion 580. As shown, the second clamping portion 540 may include an inner side 552 having an inner surface 554, an outer side 556 disposed opposite the inner side 552 and having an outer surface 558, a top side 562 (which also may be referred to as a “concave side”) having a top surface 564 (which also may be referred to as a “concave surface”), and a bottom side 566 (which also may be referred to as a “convex side”) disposed opposite the top side 562 and having a bottom surface 568 (which also may be referred to as a “convex surface”). As shown, the inner surface 554 may be a curved surface having a linear profile when viewed from the top side or the bottom side of the device 500, and the outer surface 558 may be a curved having a linear profile when viewed from the top side or the bottom side of the device 500. As shown, the top surface 564 may be a curved surface having a concave profile when viewed from one of the lateral sides of the device 500, and the bottom surface 568 may be a curved surface having a convex profile when viewed from one of the lateral sides of the device 500. According to various embodiments, the radius of curvature and the depth of curvature of the top surface 564 and the bottom surface 568 may be varied to better accommodate the anatomy of different subjects. As shown, the second clamping portion 540 may be formed as a mirror image of the first clamping portion 510 across a plane extending through the longitudinal axis A_{L} of the device 500.

During use, the occlusion device 500 may be introduced and positioned relative to a left atrial appendage while the device 500 is in the open configuration. Upon positioning, the device 500 may be transitioned from the open configuration toward the closed configuration. It will be appreciated that, with the left atrial appendage positioned between the first clamping portion 510 and the second clamping portion 540, the appendage may inhibit the device 500 from transitioning all the way to the closed configuration shown in FIGS. 5A-5C. Rather, the device 500 may transition to a deployed configuration between the open configuration and the closed configuration. One of the clamping portions 510, 540 may be positioned along the first side 132 of the left atrial appendage 120, while the other clamping portion 510, 540 may be positioned along the second side 134 of the left atrial appendage 120. When the device 500 is in the deployed configuration, the inner sides 522, 552 of the clamping portions 510, 540 may face toward the left atrial appendage 120, the outer sides 526, 556 of the clamping portions 510, 540 may face away from the left atrial appendage 120, the top sides 532, 562 of the clamping portions 510, 540 may face away from the lateral wall 112 of the left atrium 110, and the bottom sides 536, 566 of the clamping portions 510, 540 may face toward the lateral wall 112 of the left atrium 110.

It will be appreciated that use of the occlusion device 500, similar to the occlusion device 400 described above, also may alleviate the problem of incomplete occlusion of the left atrial appendage 120 due to the non-linear shape of the base 122 when the heart 100 is in situ. As compared to the clamping portions 310, 340 of the device 300 described above, the shapes of the clamping portions 510, 540 of the device 500 may better accommodate the non-linear shape of the base 122, reducing incidence of incomplete occlusion of the left atrial appendage 120 when using the occlusion device 500.

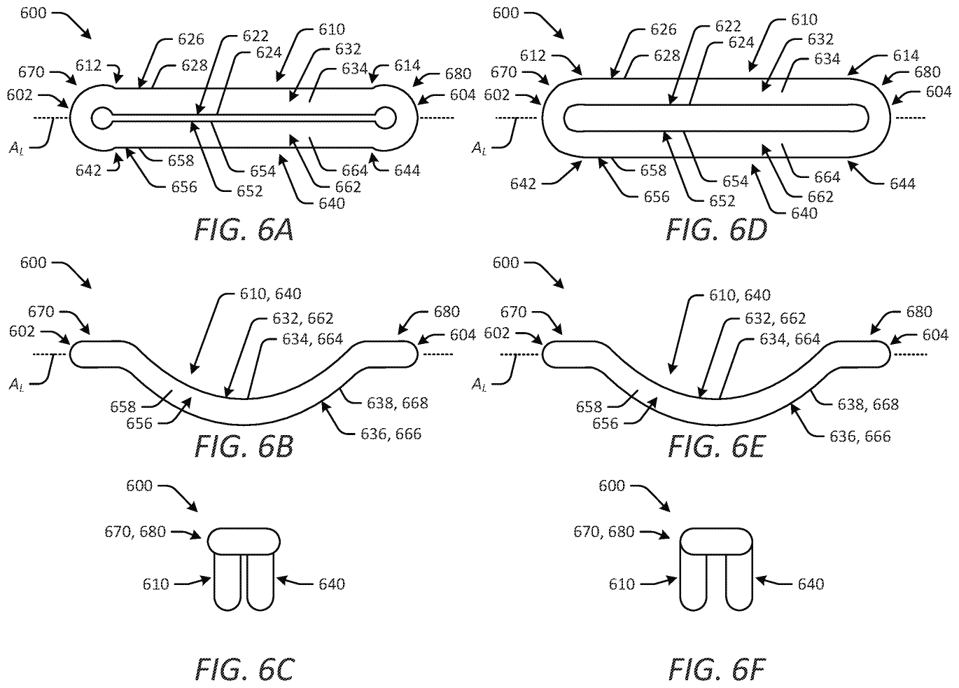

FIGS. 6A-6F illustrate another example occlusion device 600 (which also may be referred to as a “left atrial appendage occlusion device,” a “device,” a “clamp,” a “clip,” or an “implant”) in accordance with embodiments of the disclosure. Certain similarities and differences between the occlusion device 600 and the occlusion devices 400, 500 described above will be appreciated from the following description and the respective drawings. The occlusion device 600 may be formed as an elongate structure, with a first end 602 and a second end 604 disposed opposite one another along a longitudinal axis A_{L} of the device 600. As shown, the device 600 may include a first clamping portion 610 and a second clamping portion 640 connected to one another by each of a first connecting portion 670 and a second connecting portion 680. The first connecting portion 670 and the second connecting portion 680 may be configured to allow the first clamping portion 610 and the second clamping portion 640 to move relative to one another. Specifically, the occlusion device 600 may be configured to transition between a closed configuration, as shown in FIGS. 6A-6C, and an open configuration, as shown in FIGS. 6D-6F. The device 600 may be biased from the open configuration toward, or to, the closed configuration. For example, the connecting portions 670, 680 may bias the device 600 from the open configuration toward, or to, the closed configuration. As shown, the connecting portions 670, 680 may be formed as curved members configured to allow the first clamping portion 610 and the second clamping portion 640 to translate relative to one another as the device 600 transitions between the closed configuration and the open configuration.

The first clamping portion 610 may be formed as an elongate structure, with a first end 612 and a second end 614 disposed opposite one another. The first clamping portion 610 may extend from the first connecting portion 670 to the second connecting portion 680. As shown, the first clamping portion 610 may include an inner side 622 having an inner surface 624, an outer side 626 disposed opposite the inner side 622 and having an outer surface 628, a top side 632 (which also may be referred to as a “concave side”) having a top surface 634 (which also may be referred to as a “concave surface”), and a bottom side 636 (which also may be referred to as a “convex side”) disposed opposite the top side 632 and having a bottom surface 638 (which also may be referred to as a “convex surface”). As shown, the inner surface 624 may be a curved surface having a linear profile when viewed from the top side or the bottom side of the device 600, and the outer surface 628 may be a curved surface having a linear profile when viewed from the top side or the bottom side of the device 600. As shown, the top surface 634 may be a curved surface having a concave profile when viewed from one of the lateral sides of the device 600, and the bottom surface 638 may be a curved surface having a convex profile when viewed from one of the lateral sides of the device 600. According to various embodiments, the radius of curvature and the depth of curvature of the top surface 634 and the bottom surface 638 may be varied to better accommodate the anatomy of different subjects.

Similarly, the second clamping portion 640 may be formed as an elongate structure, with a first end 642 and a second end 644 disposed opposite one another. The second clamping portion 640 may extend from the first connecting portion 670 to the second connecting portion 680. As shown, the second clamping portion 640 may include an inner side 652 having an inner surface 654, an outer side 656 disposed opposite the inner side 652 and having an outer surface 658, a top side 662 (which also may be referred to as a “concave side”) having a top surface 664 (which also may be referred to as a “concave surface”), and a bottom side 666 (which also may be referred to as a “convex side”) disposed opposite the top side 662 and having a bottom surface 668 (which also may be referred to as a “convex surface”). As shown, the inner surface 654 may be a curved surface having a linear profile when viewed from the top side or the bottom side of the device 600, and the outer surface 658 may be a curved having a linear profile when viewed from the top side or the bottom side of the device 600. As shown, the top surface 664 may be a curved surface having a concave profile when viewed from one of the lateral sides of the device 600, and the bottom surface 668 may be a curved surface having a convex profile when viewed from one of the lateral sides of the device 600. According to various embodiments, the radius of curvature and the depth of curvature of the top surface 664 and the bottom surface 668 may be varied to better accommodate the anatomy of different subjects. As shown, the second clamping portion 640 may be formed as a mirror image of the first clamping portion 610 across a plane extending through the longitudinal axis A_{L} of the device 600.

During use, the occlusion device 600 may be introduced and positioned relative to a left atrial appendage while the device 600 is in the open configuration. Upon positioning, the device 600 may be transitioned from the open configuration toward the closed configuration. It will be appreciated that, with the left atrial appendage positioned between the first clamping portion 610 and the second clamping portion 640, the appendage may inhibit the device 600 from transitioning all the way to the closed configuration shown in FIGS. 6A-6C. Rather, the device 600 may transition to a deployed configuration between the open configuration and the closed configuration. One of the clamping portions 610, 640 may be positioned along the first side 132 of the left atrial appendage 120, while the other clamping portion 610, 640 may be positioned along the second side 134 of the left atrial appendage 120. When the device 600 is in the deployed configuration, the inner sides 622, 652 of the clamping portions 610, 640 may face toward the left atrial appendage 120, the outer sides 626, 656 of the clamping portions 610, 640 may face away from the left atrial appendage 120, the top sides 632, 662 of the clamping portions 610, 640 may face away from the lateral wall 112 of the left atrium 110, and the bottom sides 636, 666 of the clamping portions 610, 640 may face toward the lateral wall 112 of the left atrium 110.

It will be appreciated that use of the occlusion device 600, similar to the occlusion device 400 described above, also may alleviate the problem of incomplete occlusion of the left atrial appendage 120 due to the non-linear shape of the base 122 when the heart 100 is in situ. As compared to the clamping portions 310, 340 of the device 300 described above, the shapes of the clamping portions 610, 640 of the device 600 may better accommodate the non-linear shape of the base 122, reducing incidence of incomplete occlusion of the left atrial appendage 120 when using the occlusion device 600.

It will be appreciated that the example occlusion devices 400, 500, 600 depicted in the drawings are merely a few examples of occlusion devices in accordance with the present disclosure. In some embodiments of the disclosed occlusion devices, certain features and/or relationships illustrated in the drawings may not be present. In other embodiments of the disclosed occlusion devices, certain features and/or relationships not illustrated in the drawings may be present. In some embodiments, features of the occlusion devices 400, 500, 600 may have the relative dimensional relationships illustrated in the drawings. In other embodiments, features of the occlusion devices 400, 500, 600 may have relative dimensional relationships different from those illustrated in the drawings.

Although specific embodiments of the disclosure have been described, one of ordinary skill in the art will recognize that numerous other modifications and alternative embodiments are within the scope of the disclosure. For example, while various illustrative implementations and structures have been described in accordance with embodiments of the disclosure, one of ordinary skill in the art will appreciate that numerous other modifications to the illustrative implementations and structures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

What is claimed is:

1. An occlusion device for occluding a left atrial appendage extending from a lateral wall of a left atrium of a heart, the occlusion device comprising:
a first clamping portion configured for positioning along a first side of the left atrial appendage; and
a second clamping portion movably connected to the first clamping portion and configured for positioning along an opposite second side of the left atrial appendage while the first clamping portion is positioned along the first side;
wherein the first clamping portion and the second clamping portion each comprise:
an inner side configured to face toward the left atrial appendage when the occlusion device is in a deployed configuration;
an outer side disposed opposite the inner side and configured to face away from the left atrial appendage when the occlusion device is in the deployed configuration;
a concave side configured to face away from the lateral wall when the occlusion device is in the deployed configuration; and
a convex side disposed opposite the concave side and configured to face toward the lateral wall when the occlusion device is in the deployed configuration.

2. The occlusion device of claim 1, further comprising a connecting portion connected to the first clamping portion and the second clamping portion and positioned at a first end of the occlusion device, wherein the second clamping portion is movably connected to the first clamping portion via the connecting portion.

3. The occlusion device of claim 2, wherein the second clamping portion and the first clamping portion are not connected to one another at an opposite second end of the occlusion device.

4. The occlusion device of claim 2, wherein the occlusion device is configured to transition between an open configuration and a closed configuration.

5. The occlusion device of claim 4, wherein the connecting portion is configured to bias the occlusion device toward the closed configuration.

6. The occlusion device of claim 4, wherein the connecting portion comprises a hinge.

7. The occlusion device of claim 4, wherein the second clamping portion and the first clamping portion are configured to pivot relative to one another as the occlusion device is transitioned between the open configuration and the closed configuration.

8. The occlusion device of claim 1, further comprising:
a first connecting portion connected to the first clamping portion and the second clamping portion and positioned at a first end of the occlusion device; and
a second connecting portion connected to the first clamping portion and the second clamping portion and positioned at an opposite second end of the occlusion device;
wherein the second clamping portion is movably connected to the first clamping portion via the first connecting portion and the second connecting portion.

9. The occlusion device of claim 8, wherein the first clamping portion, the second clamping portion, the first connecting portion, and the second connecting portion form an enclosed loop defining an opening configured to receive the left atrial appendage therethrough.

10. The occlusion device of claim 8, wherein the occlusion device is configured to transition between an open configuration and a closed configuration.

11. The occlusion device of claim 10, wherein the first connecting portion and the second connecting portion are configured to bias the occlusion device toward the closed configuration.

12. The occlusion device of claim 10, wherein the first connecting portion and the second connecting portion each have a curved shape.

13. The occlusion device of claim 10, wherein the second clamping portion and the first clamping portion are configured to translate relative to one another as the occlusion device is transitioned between the open configuration and the closed configuration.

14. The occlusion device of any one of claims 1-13, wherein the inner side comprises a planar surface having a linear profile viewed from a top side or a bottom side of the occlusion device.

15. The occlusion device of any one of claims 1-13, wherein the inner side comprises a contoured surface having a contoured profile viewed from a top side or a bottom side of the occlusion device.

16. The occlusion device of any one of claims 1-13, wherein the outer side comprises a planar surface having a linear profile viewed from a top side or a bottom side of the occlusion device.

17. The occlusion device of any one of claims 1-13, wherein the outer side comprises a contoured surface having a contoured profile viewed from a top side or a bottom side of the occlusion device.

18. The occlusion device of any one of claims 1-13, wherein the concave side comprises a concave surface having a concave profile viewed from a lateral side of the occlusion device.

19. The occlusion device of claim 18, wherein the concave surface extends along at least a majority of a length of the clamping portion.

20. The occlusion device of claim 19, wherein the concave surface extends along an entirety of the length of the clamping portion.

21. The occlusion device of any one of claims 1-13, wherein the convex side comprises a convex surface having a convex profile viewed from a lateral side of the occlusion device.

22. The occlusion device of claim 21, wherein the convex surface extends along at least a majority of a length of the clamping portion.

23. The occlusion device of claim 22, wherein the convex surface extends along an entirety of the length of the clamping portion.

24. The occlusion device of any one of claims 1-13, further comprising a fabric covering extending over at least a portion of each of the first clamping portion and the second clamping portion.

25. The occlusion device of claim 24, wherein the fabric covering extends over at least a portion of each of the inner side, the outer side, the concave side, and the convex side.

26. A method for occluding a left atrial appendage extending from a lateral wall of a left atrium of a heart, the method comprising:
causing an occlusion device to transition from a closed configuration to an open configuration, wherein the occlusion device comprises a first clamping portion and a second clamping portion movably connected to the first clamping portion, and wherein the first clamping portion and the second clamping portion each comprise:
an inner side;
an outer side disposed opposite the inner side;
a concave side; and
a convex side disposed opposite the concave side;
positioning the occlusion device relative to the left atrial appendage such that the first clamping portion is positioned along a first side of the left atrial appendage and the second clamping portion is positioned along an opposite second side of the left atrial appendage; and
causing the occlusion device to transition from the open configuration to a deployed configuration in which the occlusion device occludes the left atrial appendage, wherein, when the occlusion device is in the deployed configuration, the inner sides of the first clamping portion and the second clamping portion each face toward the left atrial appendage, the outer sides of the first clamping portion and the second clamping portion each face away from the left atrial appendage, the concave sides of the first clamping portion and the second clamping portion each face away from the lateral wall, and the convex sides of the first clamping portion and the second clamping portion each face toward the lateral wall.

27. The method of claim 26, wherein the occlusion device further comprises a connecting portion connected to the first clamping portion and the second clamping portion and positioned at a first end of the occlusion device, wherein the second clamping portion is movably connected to the first clamping portion via the connecting portion.

28. The method of claim 27, wherein the second clamping portion and the first clamping portion are not connected to one another at an opposite second end of the occlusion device.

29. The method of claim 27, wherein causing the occlusion device to transition from the closed configuration to the open configuration comprises causing the first clamping portion and the second clamping portion to pivot away from one another.

30. The method of claim 27, wherein the connecting portion is configured to bias the occlusion device toward the closed configuration.

31. The method of claim 30, wherein causing the occlusion device to transition from the closed configuration to the open configuration comprises applying an external force to the occlusion device to overcome a biasing force provided by the connecting portion.

32. The method of claim 31, wherein causing the occlusion device to transition from the open configuration to the deployed configuration comprises removing the external force.

33. The method of claim 30, wherein causing the occlusion device to transition from the open configuration to the deployed configuration comprises allowing the connecting portion to bias the occlusion device to the deployed configuration.

34. The method of claim 27, wherein the connecting portion comprises a hinge.

35. The method of claim 26, wherein the occlusion device further comprises:
a first connecting portion connected to the first clamping portion and the second clamping portion and positioned at a first end of the occlusion device; and
a second connecting portion connected to the first clamping portion and the second clamping portion and positioned at an opposite second end of the occlusion device;
wherein the second clamping portion is movably connected to the first clamping portion via the first connecting portion and the second connecting portion.

36. The method of claim 35, wherein the first clamping portion, the second clamping portion, the first connecting portion, and the second connecting portion form an enclosed loop defining an opening, and wherein positioning the occlusion device relative to the left atrial appendage comprises positioning the occlusion device such that the left atrial appendage is received through the opening.

37. The method of claim 35, wherein causing the occlusion device to transition from the closed configuration to the open configuration comprises causing the first clamping portion and the second clamping portion to translate away from one another.

38. The method of claim 35, wherein the first connecting portion and the second connecting portion are configured to bias the occlusion device toward the closed configuration.

39. The method of claim 38, wherein causing the occlusion device to transition from the closed configuration to the open configuration comprises applying an external force to the occlusion device to overcome a biasing force provided by the first connecting portion and the second connecting portion.

40. The method of claim 39, wherein causing the occlusion device to transition from the open configuration to the deployed configuration comprises removing the external force.

41. The method of claim 38, wherein causing the occlusion device to transition from the open configuration to the deployed configuration comprises allowing the first connecting portion and the second connecting portion to bias the occlusion device to the deployed configuration.

42. The method of claim 35, wherein the first connecting portion and the second connecting portion each have a curved shape.

43. The method of claim 35, wherein causing the occlusion device to transition from the closed configuration to the open configuration comprises causing the first clamping portion and the second clamping portion to pivot away from one another.

44. The method of any one of claims 26-43, wherein the inner side comprises a planar surface having a linear profile viewed from a top side or a bottom side of the occlusion device.

45. The method of any one of claims 26-43, wherein the inner side comprises a contoured surface having a contoured profile viewed from a top side or a bottom side of the occlusion device.

46. The method of any one of claims 26-43, wherein the outer side comprises a planar surface having a linear profile viewed from a top side or a bottom side of the occlusion device.

47. The method of any one of claims 26-43, wherein the outer side comprises a contoured surface having a contoured profile viewed from a top side or a bottom side of the occlusion device.

48. The method of any one of claims 26-43, wherein the concave side comprises a concave surface having a concave profile viewed from a lateral side of the occlusion device.

49. The method of claim 48, wherein the concave surface extends along at least a majority of a length of the clamping portion.

50. The method of claim 49, wherein the concave surface extends along an entirety of the length of the clamping portion.

51. The method of any one of claims 26-43, wherein the convex side comprises a convex surface having a convex profile viewed from a lateral side of the occlusion device.

52. The method of claim 51, wherein the convex surface extends along at least a majority of a length of the clamping portion.

53. The method of claim 52, wherein the convex surface extends along an entirety of the length of the clamping portion.

54. The method of any one of claims 26-43, wherein the occlusion device further comprises a fabric covering extending over at least a portion of each of the first clamping portion and the second clamping portion.

55. The method of claim 54, wherein the fabric covering extends over at least a portion of each of the inner side, the outer side, the concave side, and the convex side.
